(19) United States
(12) Patent Application Publication
(10) Pub. No.: US 2004/0024256 A1
(43) Pub. Date: Feb. 5, 2004

(54) Selective halogenation of ketones

(76) Inventor: Dennis E. Jackman, Prairie Village, KS (US)

Correspondence Address: BAYER CROPSCIENCE LP, 100 BAYER ROAD, PITTSBURGH, PA, 15205, US

(21) Appl. No.: 10/186,535

(22) Filed: Jul. 1, 2002

(51) Int. Cl. C07C 45/63; B01D 3/34

(52) U.S. Cl. 568/315; 568/393; 203/28

(57) ABSTRACT

The present invention provides a process and apparatus for selectively halogenating a ketone of the formula (I)

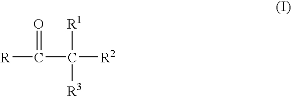

wherein R represents unsubstituted or halogen-substituted C₁-C₁₆-alkyl, or aryloxy, R¹, R² and R³ independently represent hydrogen, halogen, C₁-C₁₆-alkyl or aryl, the process involving heating the ketone to reflux in a reaction vessel such that the ketone vapor contacts a condensor attached to the reaction vessel and condenses, reacting the condensed ketone with halogen gas, and collecting the selectively halogenated ketone in the reaction vessel.

### FIELD OF THE INVENTION

This invention relates in general to halogenation reactions, and more specifically, to a process and apparatus for the selective halogenation of a ketone.

### BACKGROUND OF THE INVENTION

A complicated system for the preparation of a monohalogenated ketone is disclosed in U.S. Pat. No. 4,240,953, issued to Schubart. The ketone is vaporized and the vapors are passed through a reaction zone into a condensation zone where the vapors are condensed and caused to flow downward into a reaction device in the reaction zone. Schubart teaches several embodiments of his reaction device, with the common feature being that each provides some sort of a separate reaction container in which the condensed ketone is contacted with halogen gas.

A free radical process for the selective halogenation of ketones is taught by Barnum et al. in U.S. Pat. No. 5,449,801.

U.S. Pat. No. 4,196,150, issued to Kranz et al describes the preparation of a monochlorinated pinacolone (2-butanone, 1-chloro-3,3-dimethy) by reacting a stoichiometric excess of pinacolone (3,3-dimethyl-2-butanone) with chlorine at a temperature of −20° C. to +70° C. This product has been made, by the assignee of the present invention for about 20 years, by adding chlorine to a stream of pinacolone in a molar ratio of about 1:10. Such a process yields monochloropinacolone (MCP) to dichloropinacolone (DCP) in a ratio of about 10:1 with a majority of the starting pinacolone not reacting. The resulting mixture is subjected to a 3-phase fractional distillation wherein pinacolone and HCl are distilled off in a first column. The residue from that distillation is again distilled to remove pinacolone and some MCP. The MCP/DCP bottoms are passed through a third column to remove pure MCP and yield a residue consisting mostly of MCP and DCP.

As those skilled in the art will appreciate, the above-described process suffers from the twin disadvantages of a drastic under-chlorination of pinacolone and the subsequent loss of MCP (˜9%) in DCP formation. Those disadvantages make it necessary to process approximately 12 pounds of pinacolone to produce one pound of MCP. Such inefficiency may drastically reduce plant capacity. Further, the formation of a significant amount of DCP may represent a serious yield loss in addition to imposing a significant waste disposal cost.

To reduce or eliminate the above-described and other problems inherent in the art, therefore, a need exists for a simple process of efficiently, selectively halogenating a ketone.

### SUMMARY OF THE INVENTION

Accordingly, the present invention obviates the problems inherent in the art by providing a process for selectively halogenating a ketone of the formula (I)

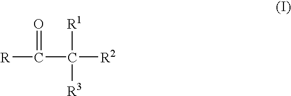

wherein

R represents unsubstituted or halogen-substituted C₁-C₁₆-alkyl, or aryloxy,

R¹, R² and R³ independently represent hydrogen, halogen or C₁-C₁₆-alkyl or aryl

the process involving heating the ketone to reflux in a reaction vessel such that the ketone vapor contacts a condensor attached to the reaction vessel and condenses, reacting the condensed ketone with halogen gas, and collecting the selectively halogenated ketone.

The present invention further provides a process for selectively halogenating a ketone of the formula (I)

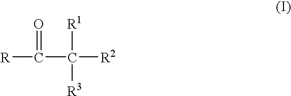

wherein

R represents unsubstituted or halogen-substituted C₁-C₁₆-alkyl, or aryloxy

R¹, R² and R³ independently represent hydrogen, halogen, C₁-C₁₆-alkyl or aryl,

the process involving heating the ketone to reflux in a reaction vessel such that the ketone vapor contacts a condensor attached by a distillation column to the reaction vessel and condenses, reacting the condensed ketone with halogen gas within the distillation column, and collecting the selectively halogenated ketone in the reaction vessel.

The present invention yet further provides an apparatus for selectively halogenating a ketone including a halogen gas container, a halogen gas feed line, a distillation column, a condenser, and a reaction vessel wherein the distillation column is attached to the reaction vessel and to the condensor so as to permit the flow therethrough of liquid and/or vapor, and wherein the distillation column is attached to the halogen gas container via the halogen gas feed line.

The present invention still further provides a process for selectively chlorinating pinacolone involving heating the pinacolone to reflux in a reaction vessel such that the pinacolone vapor contacts a condensor attached by a distillation column to the reaction vessel and condenses, reacting the condensed pinacolone with chlorine gas within the distillation column, and collecting the selectively chlorinated pinacolone in the reaction vessel.

Advantageously, the process of the present invention allows the starting ketone to be converted into the halogenated ketone without having to be recycled in a separate step or apparatus. The process of the present invention also provides a higher selectivity for the desired product, e.g., in the case of pinacolone, the ratio of MCP/DCP >14. Such selectivity increases the MCP yield from 91 to 94% and provides the ability to chlorinate almost all of the pinacolone, instead of the approximately 10% which heretofore has been possible.

These and other advantages and benefits of the present invention will be apparent from the Detailed Description of the Invention herein below.

### BRIEF DESCRIPTION OF THE FIGURE

The present invention will now be described for purposes of illustration and not limitation in conjunction with FIG. 1, which depicts one contemplated apparatus suitable for carrying out the process of the present invention.

### DETAILED DESCRIPTION OF THE INVENTION

The present invention provides a process for selectively halogenating a ketone of the formula (I)

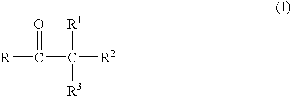

wherein

R represents unsubstituted or halogen-substituted C₁-C₁₆-alkyl, or aryloxy

R¹, R² and R³ independently represent hydrogen, halogen or C₁-C₁₆-alkyl or aryl.

The term “aryloxy” as used herein means unsubstituted or substituted phenyloxy or naphthyloxy. The term “aryl” as used herein means unsubstituted or substituted phenyl or naphthyl.

The process of the present invention involves heating the ketone to reflux in a reaction vessel such that the ketone vapor contacts a condensor attached to the reaction vessel and condenses, reacting the condensed ketone with halogen gas, and collecting the selectively halogenated ketone.

The process of the present invention may preferably be carried out in an apparatus such as that depicted in FIG. 1, wherein reaction vessel 3 may preferably be attached to reflux condensor 1 by distillation column 2. The distillation column 2 may preferably be a Vigreaux column, although other suitable fractionating columns may be used. A halogen gas feed line 4 may extend from sulfuric acid trap 6 (to visually monitor gas flow) into the distillation column 2 to provide halogen gas from the halogen gas container 5. The apparatus preferably allows the halogen gas to be added to a distillation column 2 that is constantly being flushed with distilled ketone.

Halogen gas may preferably be introduced into the flow of ketone off reflux condensor 1, preferably in darkness to avoid free radical reactions. The resulting product contains mostly the monohalogenated ketone, which migrates down into the heated reaction vessel 3 below. The liquid that subsequently forms in the reflux condenser 1 from rising vapors contains mostly starting ketone due to the much higher boiling point of the halogenated product. Therefore, most or all of the starting ketone can be converted into the desired halogenated ketone with the selectivity that may be achieved in the stream of starting material coming off the reflux condensor 1.

The equation showing this reaction is depicted below.

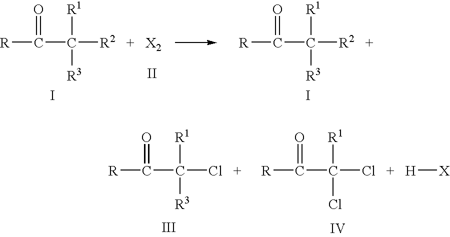

In the process of the present invention, the mole ratio of starting ketone (I) to halogen (II) is preferably much greater than one. Most of the starting ketone (I) returns to the reaction vessel 3 along with the monohalogenated ketone (III) and the dihalogenated ketone (IV). Both halogenated ketones (III) and (IV) will remain in the reaction vessel after starting ketone (I) is boiled out into the condenser 1 because starting ketone (I) has a lower boiling point than the halogenated ketones (III) and (IV). The process of the present invention preferably continues until starting ketone (I) has been mostly or completely consumed, at which time, the halogen addition is stopped. The monohalogenated ketone (III) and the dihalogenated ketone (IV) may preferably be separated by any means known to those skilled in the art. The inventor herein prefers to accomplish this separation by fractional distillation.

Use of the method of the present invention, for example in the chlorination of pinacolone, may preferably employ a chlorine addition rate of about 1 g/hr. The chlorine may preferably be added at a rate that maintains a pinacolone to chlorine molar ratio of between about 50:1 to about 1000:1. The pinacolone reflux (distillation) rate may preferably be about 1050 g/hr (molar ratio of pinacolone to chlorine may be about 750:1). Such conditions give a MCP/DCP ratio of about 13-15, which the inventor believes to be an upper limit because the same MCP/DCP ratio was observed where 1-2 g of chlorine was added to 3 moles of pinacolone in a batch chlorination. Although the method of the present invention is described herein for selectively chlorinating pinacolone, one skilled in the art will recognize that the method may be utilized to selectively add halogen(s) to other ketones.

The invention is further illustrated but is not intended to be limited by the following examples in which all parts and percentages are by weight unless otherwise specified.

### EXAMPLES

### Example 1

An attempt was made to chlorinate pinacolone (1 mol) containing some water and hydrochloric acid to monochloropinacolone (MCP) by chlorinating the reflux and vapor at the top of a Vigreaux column. The reaction was carried out in semi-darkness to prevent radical reactions. Only ˜7% dichloropinacolone (DCP) was observed where almost all of the pinacolone was chlorinated (>95%). The ratio of MCP to DCP, determined by gas chromatography, was 12:1. Only a small amount of radical chlorination occurred.

### Example 2

The process of Example 1 was repeated using a slower chlorine addition (1 g/min.), a much faster reflux rate (750 ml/min.) and no Vigreaux column. The result was a 13.7:1 ratio of MCP to DCP as determined by gas chromatography.

### Example 3

The process of Example 2 was repeated using a Vigreaux column and an oil bath at 150° C. The ratio of MCP/DCP was 15.3 as determined by gas chromatography.

### Examples 4 and 5

The process of Example 3 was repeated with a ten-fold increase in the chlorine rate (i.e., 10 g/min.). The ratio of MCP/DCP, as determined by gas chromatography, dropped to 6.3 (Ex. 4). Another reaction (Ex. 5) without the addition of a small amount of concentrated hydrochloric acid gave similar results to those seen in Example 4.

### Example 6

A catalytic amount of concentrated hydrochloric acid (1 g) was added to 1 kg (10 mol) of pinacolone. This mixture was heated to vigorous reflux in a 500 ml round bottomed flask. The vapor passed through a Vigreaux column and contacted a condenser attached to the top of the column. Chlorine gas was introduced into the condensed pinacolone within the column at a rate such that the MCP/DCP ratio, as determined by gas chromatography, was >14:1. The chlorination continued until ˜95% of the pinacolone was chlorinated. The MCP and DCP were separated by fractional distillation. About 50 g of recovered pinacolone, 1202 g of MCP and 101 g of DCP were obtained.

It will be apparent to those skilled in the art that the embodiments described herein may be modified or revised in various ways without departing from the spirit and scope of the invention. For example, it will be apparent that one could start with a monohalogenated ketone and produce a dihalogenated ketone, because the process of the present invention is so selective that only one halogen atom at a time is added to the starting ketone. The foregoing examples of the present invention are offered for the purpose of illustration and not limitation. The scope of the invention is to be measured by the appended claims.

What is claimed is:

1. A process for selectively halogenating a ketone of the formula (I) 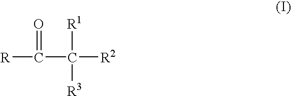
wherein
R represents unsubstituted or halogen-substituted C₁-C₁₆-alkyl, or aryloxy,
R¹, R² and R³ independently represent hydrogen, halogen, C₁-C₁₆-alkyl or aryl,
the process comprising:
heating the ketone to reflux in a reaction vessel such that the ketone vapor contacts a condenser attached to the reaction vessel and condenses;
reacting the condensed ketone with halogen gas; and
collecting the selectively halogenated ketone in the reaction vessel.

2. The process of claim 1 further including separating the selectively halogenated ketone from other reaction products.

3. The process of claim 2, wherein the separating comprises fractional distillation.

4. The process of claim 1, wherein the reaction with halogen gas occurs in at least partial darkness.

5. The process of claim 1, wherein the condenser is attached to the reaction vessel by a distillation column such that the reaction of condensed ketone with halogen gas occurs within the distillation column.

6. The process of claim 5, wherein the distillation column comprises a Vigreaux column.

7. The process of claim 1, wherein
R represents t-butyl; and
R¹, R² and R³ represent hydrogen.

8. The process of claim 7, wherein the halogen comprises chlorine.

9. An apparatus for selectively halogenating a ketone comprising:
a halogen gas container;
a halogen gas feed line;
a distillation column;
a condensor; and
a reaction vessel
wherein the distillation column is attached to the reaction vessel and to the condensor so as to permit the free flow therethrough of liquid and/or vapor, and wherein the distillation column is attached to the halogen gas container via the halogen gas feed line.

10. The apparatus of claim 9, wherein the distillation column comprises a Vigreaux column.

11. A process for selectively halogenating a ketone of the formula (I) 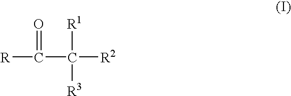
wherein
R represents unsubstituted or halogen-C₁-C₁₆-alkyl, or aryloxy,
R¹, R² and R³ independently represent hydrogen, halogen, C₁-C₁₆-alkyl or aryl,
the process comprising:
heating the ketone to reflux in a reaction vessel such that the ketone vapor contacts a condenser attached by a distillation column to the reaction vessel and condenses;
reacting the condensed ketone with halogen gas within the distillation column; and
collecting the selectively halogenated ketone in the reaction vessel.

12. The process of claim 11 further including separating the selectively halogenated ketone from other reaction products.

13. The process of claim 12, wherein the separating comprises fractional distillation.

14. The process of claim 11, wherein the reaction with halogen gas occurs in at least partial darkness.

15. The process of claim 11, wherein the distillation column comprises a Vigreaux column.

16. The process of claim 11, wherein
R represents t-butyl; and
R¹, R² and R³ represent hydrogen.

17. The process of claim 16, wherein the halogen comprises chlorine.

18. A process for selectively chlorinating pinacolone comprising:
heating the pinacolone to reflux in a reaction vessel such that the pinacolone vapor contacts a condensor attached by a distillation column to the reaction vessel and condenses;
reacting the condensed pinacolone with chlorine gas within the distillation column; and
collecting the selectively chlorinated pinacolone in the reaction vessel.

19. The process of claim 18 further including separating the selectively chlorinated pinacolone from other reaction products.

20. The process of claim 19, wherein the separating comprises fractional distillation.

21. The process of claim 18, wherein the reaction with chlorine gas occurs in at least partial darkness.

22. The process of claim 18, wherein the distillation column comprises a Vigreaux column.
